(19) United States
(12) Patent Application Publication
(10) Pub. No.: US 2026/0234200 A1
(43) Pub. Date: Aug. 13, 2026

(54) POLYPEPTIDE AND USE THEREOF IN PROMOTING MUSCLE HYPERPLASIA AND ENHANCING PHYSICAL EXERCISE PERFORMANCE

(71) Applicant: Greenyn Biotechnology Co., Ltd, Taichung City (TW)

(72) Inventors: Pang-Kuei HSU, Taichung City (TW); Yu-Cheng LIN, Taichung City (TW); Hua Chung SUN, Taichung City (TW)

(21) Appl. No.: 19/180,802

(22) Filed: Apr. 16, 2025

(30) Foreign Application Priority Data
Jul. 26, 2024 (TW) 113127720

(51) Int. Cl. C07K 14/435; A61K 38/00; A61P 21/00

(52) U.S. Cl. CPC C07K 14/43504; A61P 21/00; A61K 38/00

(57) ABSTRACT

A polypeptide and a use thereof in promoting muscle hyperplasia and enhancing physical exercise performance are provided. The polypeptide has the activity to regulate the expression of such proteins in the muscle hyperplasia pathways as p-AMPK, AKT, and mTOR. Therefore, administering an effective amount of the polypeptide or a composition containing the polypeptide to an individual can promote muscle hyperplasia in the individual and increase the individual's muscle strength, thereby producing the effect of preventing or treating diseases related to muscle damage and of enhancing physical exercise performance.

### REFERENCE TO AN ELECTRONIC SEQUENCE LISTING

The contents of the electronic sequence listing (Sequence.xml; Size: 5,222 bytes; and Date of Creation: Apr. 16, 2025) is herein incorporated by reference in its entirety.

### BACKGROUND OF THE INVENTION

### 1. Technical Field

The present invention relates to a small-molecule protein and a use thereof. More particularly, the invention relates to a polypeptide and a use thereof in promoting muscle hyperplasia and enhancing physical exercise performance.

### 2. Description of Related Art

The term “sarcopenia” refers to a disease that is characterized by continuous reduction in weight and function of a patient's skeletal muscles. The daily life or mobility of a patient with sarcopenia is likely to be affected by reduced muscle quality; for example, the patient may have problem standing or walking for a long time or lifting a heavy object, be prone to fall, or in serious cases be disabled, incapable of daily life activities, or at a higher risk of death than those without the disease. Sarcopenia not only is common among the elderly, but also occurs in young people, the main cause being insufficient protein consumption, poor nutrition, lack of regular physical exercise, or improper weight reduction.

Currently, there is no clinical drug for treating sarcopenia; patients suffering from the disease can only be advised to improve their nutrition and take more physical exercise in order to increase their muscle strength. However, most patients with sarcopenia tend to dismiss the idea of doing physical exercise, either because of a lack of muscle strength or because of poor physical exercise performance, and although the market has been supplied with a variety of nutritional supplements that are rich in protein and are claimed to be able to prevent sarcopenia, taking protein supplements can only delay muscle loss but fails to enhance muscle quality or physical exercise performance, meaning protein supplements are not effective in treating or preventing sarcopenia or its complications.

### BRIEF SUMMARY OF THE INVENTION

The primary objective of the present invention is to provide a polypeptide and a use thereof in promoting muscle hyperplasia and enhancing physical exercise performance. It is desirable that by taking the polypeptide disclosed herein on a regular basis as a supplement, muscle hyperplasia as well as an increase in muscle quality can be effectively achieved to prevent or treat diseases related to muscle loss or muscle damage.

Another objective of the present invention is to provide a polypeptide and a use thereof in promoting muscle hyperplasia and enhancing physical exercise performance, wherein the polypeptide or a composition containing the polypeptide can be administered to an individual to increase the individual's power and endurance in physical exercise.

To attain the foregoing objectives, the present invention discloses a polypeptide and a use thereof, wherein the amino acid sequence of the polypeptide includes a particular sequence or a sequence that has at least 95% homology with the particular sequence and is derived by substituting or deleting one or more amino acids of, or adding one or more amino acids to, the particular sequence, wherein the particular sequence is SEQ ID No. 1, SEQ ID No. 2, SEQ ID No. 3, SEQ ID No. 4, or SEQ ID No. 5.

In one embodiment of the present invention, the amino acid sequence of the polypeptide consists of any of the following sequences: SEQ ID No. 1, SEQ ID No. 2, SEQ ID No. 3, SEQ ID No. 4, and SEQ ID No. 5.

The polypeptide disclosed herein has the activity to promote muscle synthesis and hyperplasia. Therefore, administering an effective amount of the polypeptide disclosed herein or a composition containing the polypeptide to an individual can effectively improve or enhance the individual's muscle quality and strength.

More specifically, when the individual to which the polypeptide disclosed herein or a composition containing the polypeptide is administered has a symptom of insufficient muscle mass, the administration of an effective amount of the polypeptide or the composition to the individual can produce the effect of treating and/or preventing diseases related to insufficient muscle mass, wherein the diseases related to insufficient muscle mass include, for example, sarcopenia, bone fracture, and osteoporosis.

In another embodiment of the present invention, administering an effective amount of the polypeptide disclosed herein or a composition containing the polypeptide to an individual can effectively enhance the individual's power and endurance in physical exercise.

In addition, the polypeptide disclosed herein can be derived from an extract of a bivalve mollusk, such as an extract of blue mussel or an extract of hard clam.

### BRIEF DESCRIPTION OF THE SEVERAL VIEWS OF THE DRAWINGS

FIG. 1 is a chromatogram showing a comparison of the HPLC analysis results of different seafood extracts and of the disclosed polypeptides represented by SEQ ID No. 1-3.

FIG. 2 is a chromatogram showing a comparison of the HPLC analysis results of different seafood extracts and of the disclosed polypeptides represented by SEQ ID No. 4-5.

FIG. 3 is a chromatogram showing a comparison of the HPLC analysis results of a blue-mussel raw material, a blue mussel extract, and a blue mussel hydrolysate.

FIG. 4 shows the 1-minute, 3-minute, and 15-minute running distances of each group of mice.

FIG. 5 shows the 1-minute, 3-minute, and 15-minute running speeds of each group of mice.

FIG. 6 shows the grip strength of each group of mice.

FIG. 7 shows the total daily running distance of each group of mice during the animal test.

FIG. 8 shows the serum testosterone levels of certain groups of mice.

FIG. 9 shows the changes in each test subject's muscle mass during the 14-day human subject research.

FIG. 10 shows the total grip strength of each test subject's left and right hands.

### DETAILED DESCRIPTION OF THE INVENTION

The present invention discloses a polypeptide and a use thereof in promoting muscle hyperplasia and enhancing physical exercise performance. The polypeptide disclosed herein is a small molecule composed of 9-12 amino acids and has the activity to regulate the expression of such proteins in the muscle hyperplasia pathways as p-AMPK, AKT, and mTOR. Therefore, administering an effective amount of the polypeptide or a composition containing the polypeptide to an individual can promote muscle hyperplasia in the individual and increase the individual's muscle strength, thereby producing the effect of preventing or treating diseases related to muscle damage and of enhancing physical exercise performance.

When the individual is a human, the effective amount of the composition is at least 300-600 mg/day.

In one embodiment of the present invention, the sequence of the polypeptide is as represented by one of SEQ ID No. 1-SEQ ID NO. 5.

In another embodiment of the present invention, the sequence of the polypeptide is a sequence having at least 95% homology with any of SEQ ID No. 1-SEQ ID NO. 5. For example, the sequence of the polypeptide is a derivative obtained by performing one or a combination of the following: substituting one or more amino acids of any of SEQ ID No. 1-SEQ ID NO. 5, adding one or more amino acids to any of SEQ ID No. 1-SEQ ID NO. 5, deleting one or more amino acids of any of SEQ ID No. 1-SEQ ID NO. 5, and modifying one or more amino acids of any of SEQ ID No. 1-SEQ ID NO. 5.

In one embodiment of the present invention, the polypeptide can be prepared by an artificial synthesis method well known in the art or be produced by a bio-production platform. More specifically, a biotechnology well known in the art can be used to introduce into a recombinant vector a nucleic acid molecule that can produce the polypeptide through transcription, and then the recombinant vector is transferred into a host cell in order to be expressed, thereby producing an intermediate product containing the polypeptide. The polypeptide is subsequently derived from the intermediate product by a purification and separation technique.

In another embodiment of the present invention, the polypeptide disclosed herein can be derived from an extract of a bivalve mollusk, such as an extract of blue mussel, and the bivalve mollusk extract is obtained by performing an extraction process on a specific bivalve mollusk using a predetermined extraction solvent, wherein the extraction process may employ a solvent extraction method, an ultrasonic extraction method, a supercritical extraction method, or a combination of at least two of the above, and wherein the extraction solvent may be water, ethanol, methanol, or another solvent well known in the art.

In another embodiment of the present invention, the polypeptide disclosed herein can be derived from a hydrolysate of a bivalve mollusk, such as a hydrolysate of blue mussel, and the bivalve mollusk hydrolysate is obtained by performing a hydrolysis process with a hydrolytic enzyme, wherein the hydrolytic enzyme may be an alkaline protease or another hydrolytic enzyme that is well known in the art and acceptable in the food industry.

In one embodiment of the present invention, the aforesaid composition includes a polypeptide represented by one of SEQ ID No. 1, SEQ ID No. 2, SEQ ID No. 3, SEQ ID No. 4, and SEQ ID No. 5.

In another embodiment of the present invention, the composition includes polypeptides each represented by one of SEQ ID No. 1, SEQ ID No. 2, SEQ ID No. 3, SEQ ID No. 4, and SEQ ID No. 5.

The term “blue mussel,” also known as “mussel,” refers to a bivalve mollusk that can be consumed as seafood.

The term “blue mussel powder” refers to a product obtained by cooking and shelling blue mussel, drying the cooked and shelled mussel, and grinding the dried mussel into powder.

The term “sarcopenia” refers to a disease that is characterized by continuous reduction in weight and function of a patient's skeletal muscles. Clinically, sarcopenia is assessed according to an individual's ability to lift a heavy object, whether or not an aid is needed for walking, the degree of difficulty of standing from a seated position on a chair, the number of steps that the individual can go up, and the number of times of fall, among others.

The term “complications of sarcopenia” refers to the diseases or symptoms caused by sarcopenia, such as disability, reduced physical strength/stamina, osteoporosis, bone fracture, cognitive impairment, metabolic syndrome, and cardiovascular diseases.

The term “effective amount” refers to the dose/dosage of intake that is required to prevent, improve, or treat a disease or symptom in question.

The term “treat” refers to improving an individual's disease or symptom in question, or delaying the progression of the disease, by administering the polypeptide disclosed herein or a composition containing the polypeptide to the individual.

The term “prevent” refers to keeping a disease in question from occurring in an individual at risk of contracting the disease, or delaying the time point of occurrence of the disease in the individual, by administering the polypeptide disclosed herein or a composition containing the polypeptide to the individual.

The term “composition” refers to a substance whose main active ingredient is the polypeptide disclosed herein or is a substance containing the polypeptide, and which can be prepared in different product types (e.g., a nutritional supplement, food, drug, or drink) as needed and in different dosage forms (e.g., tablet, powder sachet, or jelly) according to such factors as the product type, the individuals to which the composition is to be administered, and marketing considerations.

To better expound the technical features of the present invention and their effects, some experimental examples are described below.

The cells used in the following examples are cells easily obtainable by a person of ordinary skill in the art and therefore need not be deposited for patent application purposes.

The animal test in the following examples complied with applicable ethical guidelines on animal tests.

The numbers given below, be they the numerical values of operation conditions, experimental data, or dosages, serve only as examples of the present invention and do not limit the scope of patent protection or of interpretation of the invention. Moreover, it is understood that the data or numbers disclosed in the following examples have acceptable ranges of errors as can be understood by a person of ordinary skill in the art according to general common knowledge or experimental conventions. Generally, in the field to which the invention pertains, an acceptable range of errors of a disclosed numerical value is ±10% of the numerical value. For example, an acceptable range of errors of the temperature 37° C. is 37° C.±3.7° C.

The sample doses used in the following animal test were exemplary only. In other words, the doses used in the animal test can be changed according to the species of the recipients and the conditions thereof and are not restrictive of the scope of patent protection of the present invention.

### Example 1: Preparation of Synthetic Sequences

Polypeptides represented by SEQ ID No. 1-SEQ ID NO. 5 were artificially synthesized, and the sequences of the polypeptides prepared were verified, as shown in Table 1.

TABLE 1
Data of each polypeptide

| Abbreviated | | Amino acid | Molecular |
|---|---|---|---|
| name | Sequence ID no. | sequence | weight |
| VQ-11 | SEQ ID No. 1 | VDDSDFFPFPQ | 1042.4 |
| AQ-11 | SEQ ID No. 2 | AGPGDPSKPTQ | 1053.5 |
| SS-12 | SEQ ID No. 3 | SNQPDQVNGNAS | 1230.5 |
| GE-11 | SEQ ID No. 4 | GIQGPSGPQGE | 1026.2 |
| SR-9 | SEQ ID No. 5 | SDYDNLNAR | 1067.1 |

### Example 2: Cell Test

C2C12 myoblasts were inoculated into a 24-well plate at a concentration of 1×10⁴ counts/mL and were cultivated in the following culture solution in a 37° C., 5% carbon dioxide environment, with the culture solution replaced every 2-3 days.

Culture solution: A Dulbecco's modified Eagle medium (DMEM) containing 10% fetal bovine serum, optionally added with an antibiotic such as penicillin (at a final concentration of 10.0 IU/mL) or streptomycin (at a final concentration of 10.0 IU/mL).

When the cultivated C2C12 myoblasts reached 90% confluence, the culture solution was changed to a DMEM containing 2% equine serum, and then the cultivation continued, with the culture solution replaced every 48 hours. After cultivation for a total of 4 days, the C2C12 myoblasts were found to have differentiated into myotubes and were therefore ready to be tested in groups.

The C2C12 myoblasts were divided into the following groups and then cultured under the condition specified for each group:
Group 1 was the blank group.
Group 2 was the positive control group and was added with 100 nM insulin.
Groups 3 were the sample groups and were each added with a sample at 400 μg/mL, wherein the sample was the polypeptide of SEQ ID No. 1, No. 2, No. 3, No. 4, or No. 5 in example 1.

After cultivation for 24 hours, cells were collected from each group, and protein extraction was performed on the cells collected from each group. The expression of each of the following proteins was then analyzed by the western blot method: 5′ adenosine monophosphate-activated protein kinase (AMPK), phosphorylated AMPK (p-AMPK), mammalian target of rapamycin (mTOR), phosphorylated mTOR (p-mTOR), protein kinase B (AKT), and phosphorylated AKT (p-AKT). Following that, the expression ratios p-AMPK/AMPK, p-AKT/AKT, and p-mTOR/mTOR were each calculated as a multiple of the corresponding ratio of the blank group, to which no sample was added. The results are shown in Table 2, with the p-AMPK/AMPK value, the p-AKT/AKT value, and the p-mTOR/mTOR value of the blank group being 1.

TABLE 2
Protein analysis results corresponding to each polypeptide

| | | p-AMPK/ | | p-mTOR/ |
|---|---|---|---|---|
| | | AMPK | p-AKT/AKT | mTOR |
| Abbreviated | Sequence | (number of | (number of | (number of |
| name | ID no. | multiples) | multiples) | multiples) |
| VQ-11 | SEQ ID No. 1 | 1.01 | 0.63 | 1.10 |
| AQ-11 | SEQ ID No. 2 | 1.24 | 0.58 | 1.32 |
| SS-12 | SEQ ID No. 3 | 0.88 | 0.56 | 1.46 |
| GE-11 | SEQ ID No. 4 | 1.85 | 0.60 | 0.91 |
| SR-9 | SEQ ID No. 5 | 0.66 | 1.58 | 1.41 |

It can be known from the results in Table 2 that the polypeptides disclosed herein had the activity to increase the expression of such proteins as p-AMPK, AKT, and mTOR. Moreover, it is well known to a person of ordinary skill in the art that p-AMPK, AKT, and mTOR are the main muscle hyperplasia regulating factors in the muscle hyperplasia mechanism. Therefore, given that the foregoing results have proved that each of the polypeptides disclosed herein (whose sequences are represented separately by SEQ ID No. 1-5) has the activity to enhance p-AMPK, AKT, and mTOR expression, administering an effective amount of any of the polypeptides disclosed herein (whose sequences are represented separately by SEQ ID No. 1-5) to an individual can promote myogenesis, the decomposition of damaged muscles, and muscle hyperplasia, thereby producing the effect of preventing or treating sarcopenia or diseases related thereto or of enhancing physical exercise performance.

### Example 3: Preparation of Seafood Extracts

This example was intended to verify that the polypeptides disclosed herein (each of which has a sequence represented by one of SEQ ID No. 1-5) exist in a blue mussel extract prepared by a predetermined preparation process, as detailed below.

0.3 g of blue mussel powder was mixed into 3 mL of double-distilled water (ddH₂O) to form an aqueous blue mussel solution (3 mL). The aqueous blue mussel solution was subjected to ultrasonic extraction for about 1 hour and then centrifuged (at 1000 g), before the supernatant was separated and filtered to obtain a blue mussel extract solution.

20 μL of the blue mussel extract solution was analyzed by high-performance liquid chromatography (HPLC) under the following conditions:

For polypeptides whose sequences are represented separately by SEQ ID No. 1-3, the analysis conditions included the use of a C18 column and a mobile phase containing C, which was acetonitrile+0.05% trifluoroacetic acid (TFA), and D, which was double-distilled water+0.05% TFA, with the flow velocity being 1.0 mL/min, and the ratios between the C phase and the D phase as shown in Table 3:

TABLE 3
HPLC analysis conditions (1)

| Time (min) | % C | % D |
|---|---|---|
| 5.00 | 1.0 | 99.0 |
| 20.00 | 5.0 | 95.0 |
| 35.00 | 10.0 | 90.0 |
| 40.00 | 50.0 | 50.0 |
| 45.00 | 1.0 | 99.0 |

For polypeptides whose sequences are represented separately by SEQ ID No. 4-5, the analysis conditions included the use of a C18 column and a mobile phase containing C, which was acetonitrile+0.05% TFA, and D, which was double-distilled water+0.05% TFA, with the flow velocity being 1.0 mL/min, and the ratios between the C phase and the D phase as shown in Table 4:

TABLE 4
HPLC analysis conditions (2)

| Time (min) | % C | % D |
|---|---|---|
| 0 | 8 | 92 |
| 3 | 14 | 86 |
| 18 | 16 | 84 |
| 28 | 21 | 79 |
| 38 | 25 | 75 |
| 40 | 25 | 75 |
| 45 | 50 | 50 |
| 50 | 8 | 92 |

The aforesaid preparation method was also used to prepare extracts of abalone, blood clam, hard clam, oyster, and Corbicula (in two batches), and the extracts obtained received the foregoing HPLC analyses, too.

In addition, each of the polypeptides disclosed herein (whose sequences are represented separately by SEQ ID No. 1-5) was analyzed under the corresponding HPLC analysis conditions.

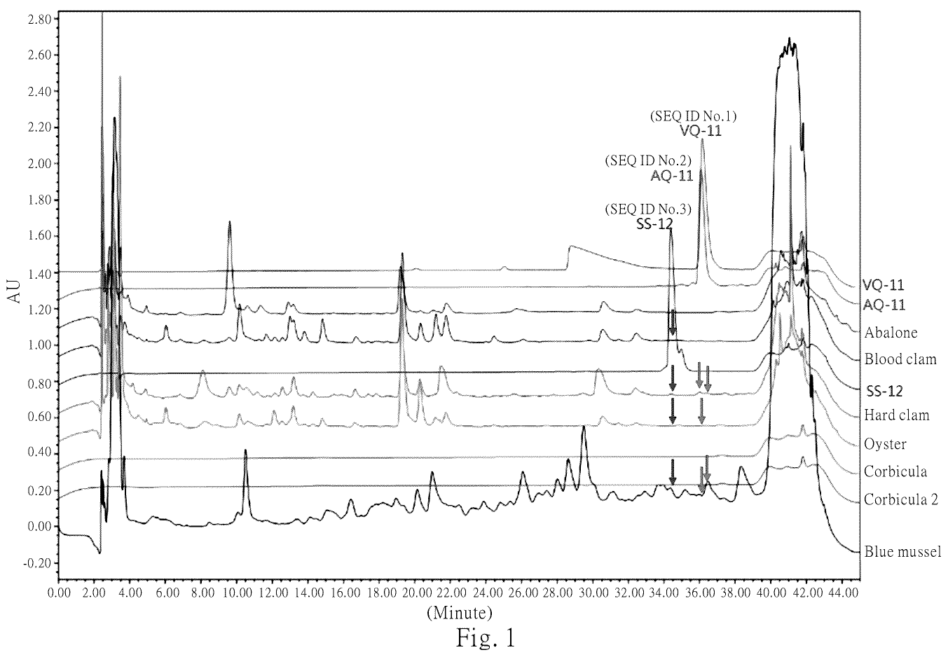

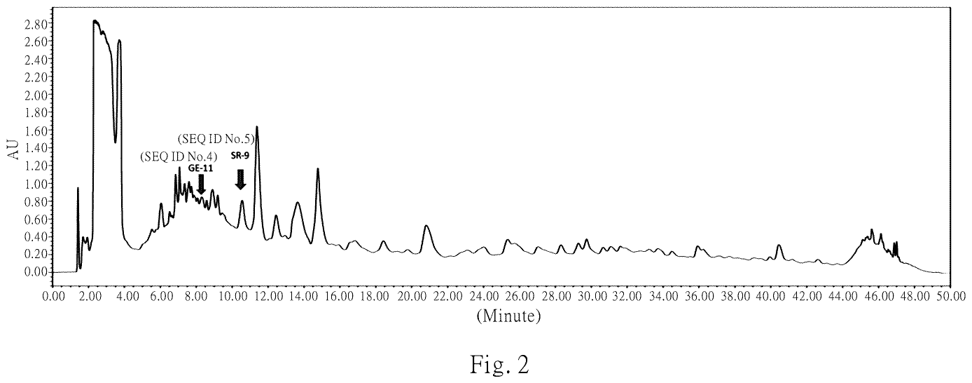

The chromatograms obtained from all the HPLC analyses are compared in FIG. 1 and FIG. 2. It can be known from the results in FIG. 1 that only the hard clam extract and the blue mussel extract contained the polypeptides disclosed herein whose sequences are represented separately by SEQ ID No. 1-3. It can be known from the results in FIG. 2 that only the blue mussel extract contained the polypeptides disclosed herein whose sequences are represented separately by SEQ ID No. 4-5, with the retention time of the polypeptide whose sequence is represented by SEQ ID No. 4 being about 8.5 minutes, and the retention time of the polypeptide whose sequence is represented by SEQ ID No. 5 being about 10.9 minutes. The foregoing results indicate that the polypeptides disclosed herein (each having a sequence represented by one of SEQ ID No. 1-5) are unique, are not widely present in bivalve mollusks or fish, and are obtainable only through a specific extraction process.

### Example 4: Analysis of the Ingredients of Blue Mussel

A blue mussel extract was prepared by the method of example 3, and a blue mussel hydrolysate was obtained by hydrolyzing blue mussel with an alkaline protease.

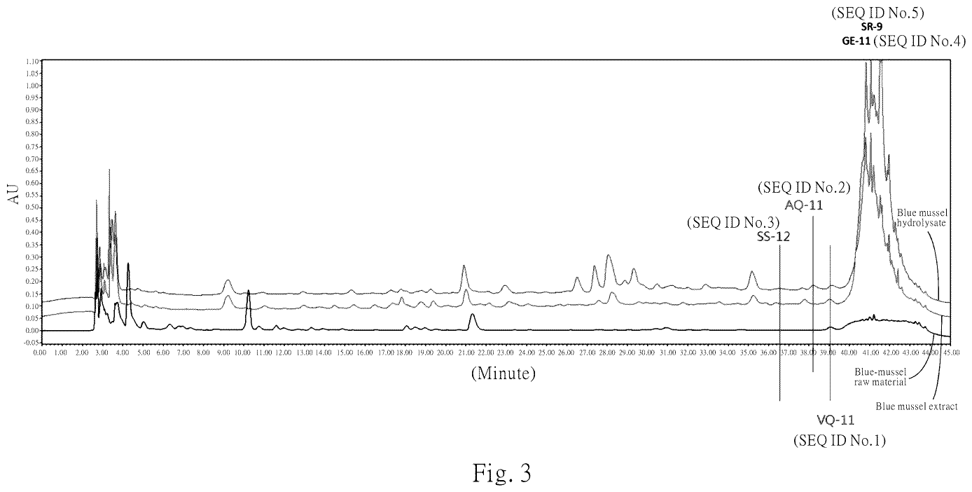

The blue-mussel raw material (i.e., blue mussel powder), the blue mussel extract, and the blue mussel hydrolysate were analyzed by HPLC to determine whether any of them contained polypeptides whose sequences are represented separately by SEQ ID No. 1-5, and the results are shown in FIG. 3. It can be inferred from the results in FIG. 3 that blue mussel does contain the polypeptides disclosed herein (each having a sequence represented by one of SEQ ID No. 1-5).

### Example 5: Animal Test

A plurality of 10-week-old male ICR mice were randomly divided into 5 groups, and the test lasted for 4 weeks.

In week 1 of the test, each group of mice received physical exercise training for 3 times, wherein the physical exercise training included a running test and a grip strength test.

During weeks 2-4 of the test, the mice were fed as follows:
Group 1: Not fed with any sample.
Group 2: Fed with taurine at a dose of 300 mg/kg-body weight.
Group 3: Fed with a blue mussel extract at a dose of 45 mg/kg-body weight.
Group 4: Fed with the blue mussel extract at a dose of 90 mg/kg-body weight.
Group 5: Fed with the blue mussel extract at a dose of 180 mg/kg-body weight.

The blue mussel extract administered to the mice in groups 3-5 was prepared by the method disclosed in example 3 and contained polypeptides each having a sequence represented by one of SEQ ID No. 1-5.

After rearing the mice in each group under the corresponding conditions, physical exercise indicators were determined. The physical exercise indicator tests included:

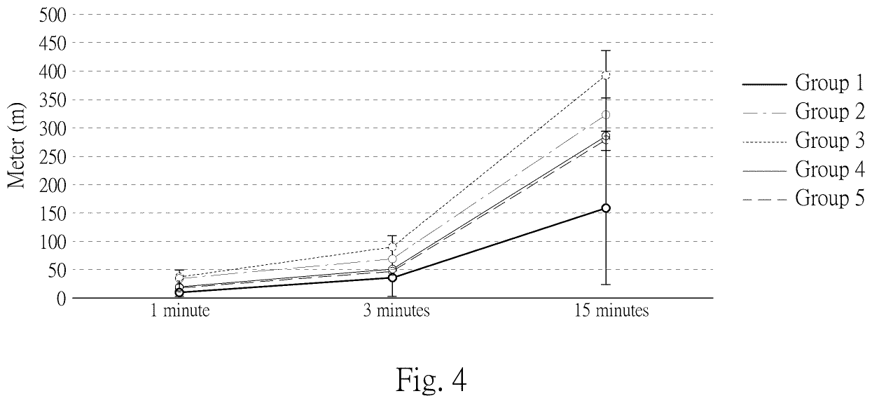

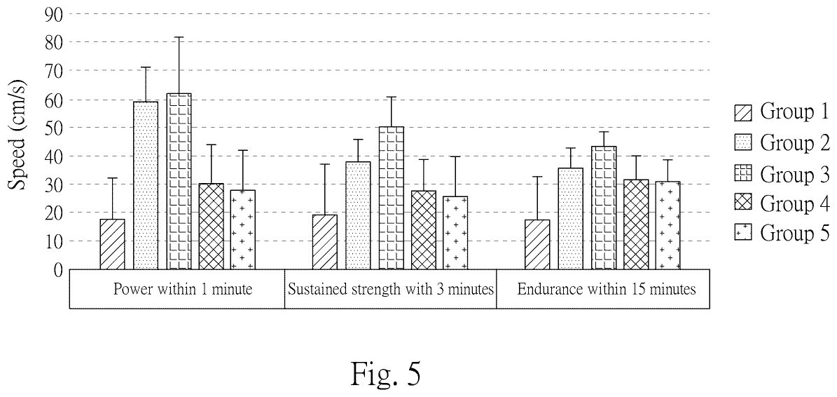

Making the mice in each group run on a running wheel for 15 minutes, with the running distance and running speed measured at the end of 1 minute, 3 minutes, and 15 minutes, the results being shown in FIG. 4 and FIG. 5;

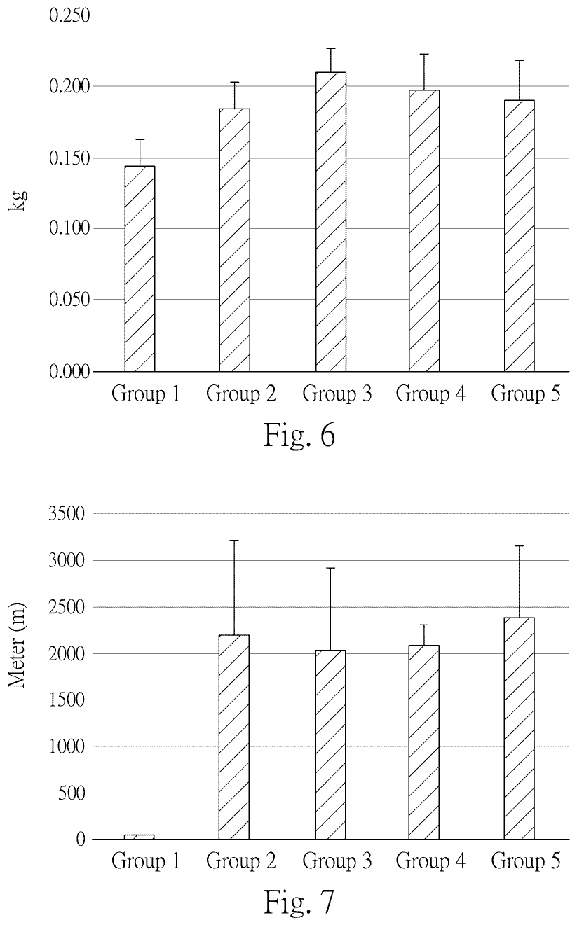

Hanging the mice in each group from a net by their claws, pulling the mice vertically downward, and recording the grip strength of each mouse at the instant when the mouse's claws let go of the net, the results being shown in FIG. 6; and

Recording the cumulative running distances of the mice in each group during the test, the results being shown in FIG. 7.

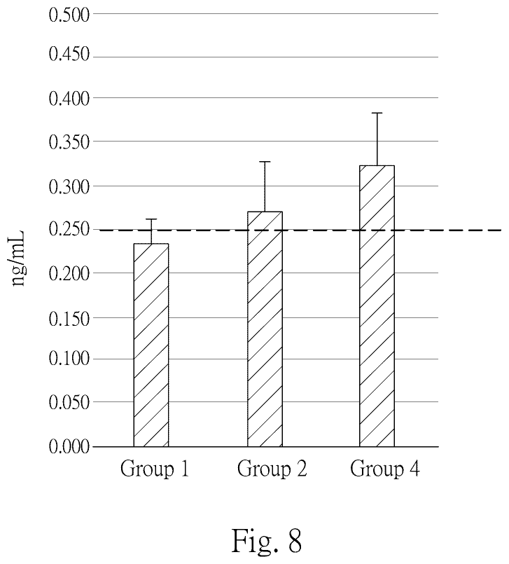

Furthermore, blood was collected from the mice in group 1, group 2, and group 4, and the serum testosterone levels of the collected blood were measured. The results are shown in FIG. 8.

It can be known from the results in FIG. 4 to FIG. 7 that administering the blue mussel extract disclosed herein led to significant enhancements of physical exercise performance in comparison with the mice in group 1. When it comes to enhancements of the power in physical exercise and of physical exercise performance, the mice in group 5 performed significantly better than the mice in group 1, regardless of whether the results were measured at the end of 1 minute, 3 minutes, or 15 minutes; and although not outperforming the mice in group 2, the mice in group 3 and group 4 still managed to maintain their performance effectively and performed better than the mice in group 1 continuously for a long time (see FIG. 4 and FIG. 5). As for the grip strength, the mice in group 3 to group 5 performed significantly better than the mice in group 2, with the mice in group 3 having the highest grip strength (see FIG. 6). As for the daily running distance, the mice in group 3 and group 4 had similar performance to the mice in group 2, and the mice in group 5 outperformed the mice in group 2 (see FIG. 7).

Besides, it can be known from the results in FIG. 8 that the blue mussel extract disclosed herein effectively increased the serum testosterone level in the mice in group 4, and this indicates that the blue mussel extract disclosed herein can produce the effect of enhancing physical exercise performance.

The results presented above demonstrate that administering the blue mussel extract disclosed herein to an individual can effectively enhance the individual's physical exercise performance and keep the individual's physical exercise performance at the enhanced level, regardless of whether the dose administered is low, medium, or high; and that the higher the dose administered, the greater the enhancement of the individual's physical exercise performance.

As proven by the results of example 3 and example 5, administering any of the polypeptides disclosed herein (whose sequences are represented separately by SEQ ID No. 1-5) or a composition containing polypeptides whose sequences are represented separately by SEQ ID No. 1-5 (e.g., a blue mussel extract) to an individual can improve the individual's physical exercise performance and muscle mass; that is to say, the polypeptides disclosed herein (each having a sequence represented by one of SEQ ID No. 1-5) or a composition containing the same can be used to treat or prevent sarcopenia or its complications and enhance physical exercise performance.

### Example 6: Human Subject Research

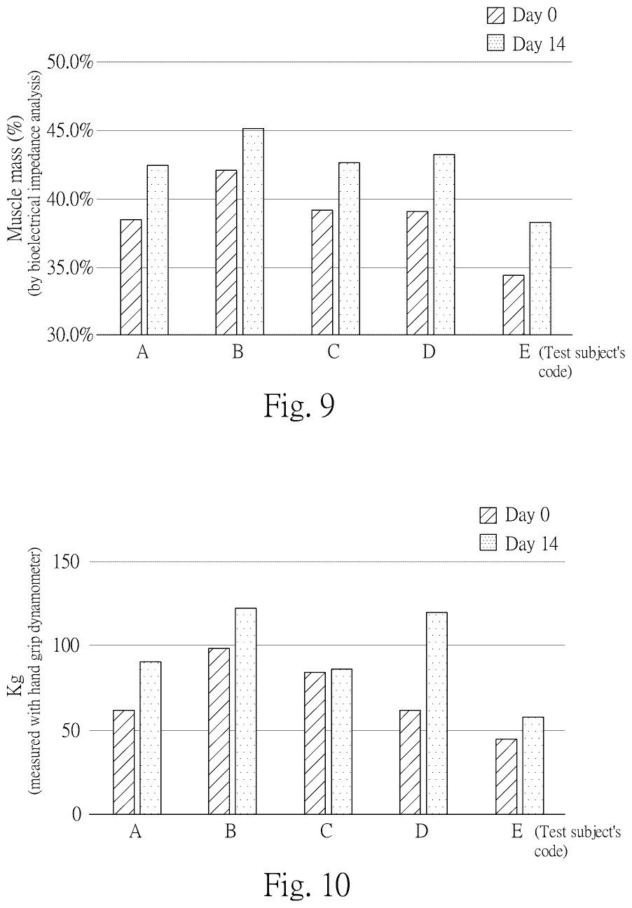

There were a total of 5 test subjects. 600 mg of blue mussel extract was administered to each test subject in the morning, wherein the blue mussel extract was prepared by the method disclosed in example 3 and contained polypeptides each having a sequence represented by one of SEQ ID No. 1-5. After administration of the blue mussel extract for 14 days in a row, each test subject's muscle mass ratio (i.e., muscle weight/body weight) and the total grip strength of each test subject's left and right hands were measured. The results are shown in FIG. 9 and FIG. 10.

It can be known from the results in FIG. 9 and FIG. 10 that administering the blue mussel extract disclosed herein for 14 days increased muscle mass by about 9.7% and increased the grip strength of both hands by about 24%. The results indicate that the polypeptides disclosed herein (each having a sequence represented by one of SEQ ID No. 1-SEQ ID No. 5) or a composition containing the same (e.g., a blue mussel extract) is indeed capable of increasing an individual's physical exercise performance, muscle mass, and muscle strength. Therefore, the polypeptides disclosed herein (each having a sequence represented by one of SEQ ID No. 1-SEQ ID No. 5) or a composition containing the same can be used to treat or prevent sarcopenia or its complications and enhance physical exercise performance.

1. A polypeptide having an amino acid sequence comprising either a particular sequence or a sequence that has at least 95% homology with the particular sequence and is derived by substituting or deleting one or more amino acids of, or adding one or more amino acids to, the particular sequence, wherein the particular sequence is selected from the group consisting of SEQ ID No. 1, SEQ ID No. 2, SEQ ID No. 3, SEQ ID No. 4, and SEQ ID No. 5.

2. The polypeptide of claim 1, wherein the amino acid sequence is SEQ ID No. 1, SEQ ID No. 2, SEQ ID No. 3, SEQ ID No. 4, or SEQ ID No. 5.

3. The polypeptide of claim 1, wherein the polypeptide is separated from a bivalve mollusk extract.

4. The polypeptide of claim 3, wherein the bivalve mollusk extract is derived from blue mussel.

5. A method for preventing and/or treating sarcopenia or enhancing physical exercise performance, the method comprising: administering an effective amount of a polypeptide composition to an individual, wherein the polypeptide composition comprises the polypeptide of claim 1.

6. The method for preventing and/or treating sarcopenia or enhancing physical exercise performance as claimed in claim 5, wherein the polypeptide composition comprises at least one polypeptide which amino acid sequence is SEQ ID No. 1, SEQ ID No. 2, SEQ ID No. 3, SEQ ID No. 4, or SEQ ID No. 5.

7. The method for preventing and/or treating sarcopenia or enhancing physical exercise performance as claimed in claim 6, wherein the polypeptide composition comprises polypeptides each represented by one of SEQ ID No. 1, SEQ ID No. 2, SEQ ID No. 3, SEQ ID No. 4, and SEQ ID No. 5.

8. The method for preventing and/or treating sarcopenia or enhancing physical exercise performance as claimed in claim 6, wherein the polypeptide is separated from a bivalve mollusk extract.

9. The method for preventing and/or treating sarcopenia or enhancing physical exercise performance as claimed in claim 5, wherein the polypeptide composition comprises a bivalve mollusk extract.

10. The method for preventing and/or treating sarcopenia or enhancing physical exercise performance as claimed in claim 9, wherein the bivalve mollusk extract is obtained by performing ultrasonic extraction on blue mussel, with water serving as an extraction solvent.


SEQUENCE LISTING (5 sequences)

SEQ ID NO: 1 - protein, 11 aa, synthetic construct
VDDSDFFPFPQ

SEQ ID NO: 2 - protein, 11 aa, synthetic construct
AGPGDPSKPTQ

SEQ ID NO: 3 - protein, 12 aa, synthetic construct
SNQPDQVNGNAS

SEQ ID NO: 4 - protein, 11 aa, synthetic construct
GIQGPSGPQGE

SEQ ID NO: 5 - protein, 9 aa, synthetic construct
SDYDNLNAR